(19) United States
(12) Patent Application Publication
(10) Pub. No.: US 2023/0263639 A1
(43) Pub. Date: Aug. 24, 2023

(54) INTERVERTEBRAL FUSION CAGE

(71) Applicant: BEIJING ZHISU HEALTH TECHNOLOGY CO. LTD, Beijing (CN)

(72) Inventors: Jing ZHANG, Beijing (CN); Kai XU, Beijing (CN); Xiaoyu WEN, Beijing (CN); Lu SUN, Beijing (CN); Xuewei MA, Beijing (CN); Hao LIU, Beijing (CN)

(21) Appl. No.: 18/024,001

(22) PCT Filed: Jul. 2, 2021
(86) PCT No.: PCT/CN2021/104203

§ 371 Date: Feb. 28, 2023

(30) Foreign Application Priority Data
Aug. 28, 2020 (CN) 202010885444.3

(51) Int. Cl. A61F 2/44

(52) U.S. Cl. CPC A61F 2/447; A61F 2002/30784

(57) ABSTRACT

An intervertebral fusion cage (1). The intervertebral fusion cage (1) is provided with a filler hole (11), which runs through both ends of the intervertebral fusion cage (1), and a plurality of pore areas (14) with spacings are provided on a side face of the intervertebral fusion cage (1). By means of a reasonable arrangement of the pore areas (14), a spacing is provided between the adjacent pore areas (14) so as to ensure that the intervertebral fusion cage (1) has a sufficient strength while having a predetermined elastic modulus, thereby preventing the intervertebral fusion cage (1) from being cracked or even broken when being subjected to an external force, which facilities solution of a technical problem of an insufficient strength of an intervertebral fusion cage (1) due to pore structures in the prior art.

### TECHNICAL FIELD

The invention relates to the field of medical instruments, and in particular to an intervertebral fusion cage.

### BACKGROUND ART

At present, an intervertebral fusion cage is an implantable medical instrument that is applied between upper and lower vertebrae of a human body, and is one of main implants for achieving a space fusion of adjacent vertebrae of a spine, and safety and effectiveness thereof directly affect an effect of a bony fusion of adjacent vertebral bodies. Due to different individual situations (such as bone densities, positions and structures of adjacent vertebrae during implantation, and so on), medical instruments with different elastic moduli are required; and only when the elastic modulus of the intervertebral fusion cage is close to the bone elastic modulus of the human body may problems such as stress shielding be avoided. So in order that the elastic modulus of the intervertebral fusion cage may be consistent with the corresponding bone elastic modulus, some pore structures are generally used to make the intervertebral fusion cage have the expected elastic modulus.

However, in the prior art, due to an unreasonable arrangement of the pore areas, the strength of the intervertebral fusion cage is generally insufficient. In some cases, such as after the intervertebral fusion cage is implanted in a human body, the vertebrae will exert an external force against the intervertebral fusion cage during the movement of the human body, which may in tum cause the intervertebral fusion cage having the insufficient strength to be cracked or even completely broken, and the fragments of the cracked or broken intervertebral fusion cage will be very dangerous if they are left in the human body.

Thus, researchers are looking for such an intervertebral fusion cage that when it has pore areas to achieve the required elastic modulus, the strength of the intervertebral fusion cage may be ensured, thereby preventing the intervertebral fusion cage from being cracked or even broken when being subjected to an external force, which facilities solution of a technical problem of an insufficient strength of an intervertebral fusion cage due to pore areas in the prior art.

### SUMMARY OF THE INVENTION

The application provides an intervertebral fusion cage, and the object thereof lies in obtaining a strength satisfying a requirement while obtaining a required elastic modulus by means of a reasonable arrangement of pore areas, thereby preventing the intervertebral fusion cage from being cracked or even broken when being subjected to an external force.

The intervertebral fusion cage is provided with a filler hole, which runs through both ends of the intervertebral fusion cage; and a plurality of pore areas with spacings are provided on a side face of the intervertebral fusion cage.

In an embodiment, the both ends of the intervertebral fusion cage are a first end face and a second end face, respectively; wherein at least one of the pore areas extends towards the both ends, and is connected to the first end face and the second end face, respectively.

In an embodiment, the side face is formed by successively connecting a first side face and a second side face as well as a third side face and a fourth side face and forming a closure;

the plurality of pore areas include a first pore area and a second pore area; and
the first pore area and the second pore area are provided on the first side face and the third side face, respectively.

In an embodiment, the second side face is provided with an instrument hole.

In an embodiment, the plurality of pore areas further include a third pore area; and

the third pore area is provided on the fourth side face.

In an embodiment, the first side face and the second side face as well as the third side face and the fourth side face transition by rounded corners.

In an embodiment, there is an inclination angle between the first end face and the second end face.

In an embodiment, a first protrusion and a second protrusion are provided on the first end face and/or the second end face, respectively.

In an embodiment, there is a side face angle between the first side face and the third side face.

In an embodiment, the pore area is a crystal structure area with pores that is constructed by connecting rod supports.

As may be seen, based on the aforesaid embodiments, the present application provides an intervertebral fusion cage, where by means of a reasonable arrangement of the pore structure areas, it may be ensured that the intervertebral fusion cage has a sufficient strength while having a predetermined elastic modulus, thereby preventing the intervertebral fusion cage from being cracked or even broken when being subjected to an external force, which facilities solution of a technical problem of an insufficient strength of an intervertebral fusion cage due to pore structures in the prior art.

### BRIEF DESCRIPTION OF THE DRAWINGS

FIG. 1 is a schematic diagram of an overall structure of the intervertebral fusion cage of the invention in an embodiment;

FIG. 2 is a schematic diagram of an overall structure of the intervertebral fusion cage of the invention in another embodiment;

FIG. 3 is a stress analysis diagram of an arrangement state 100 of pore areas of the intervertebral fusion cage;

FIG. 4 is a stress analysis diagram of an arrangement state 200 of pore areas of the intervertebral fusion cage;

FIG. 5 is a schematic diagram of a side view of a structure of one side of the intervertebral fusion cage of the invention;

FIG. 6 is a schematic diagram of a front view of the structure of the intervertebral fusion cage of the invention;

FIG. 7 is a schematic diagram of a rear view of a back face of the intervertebral fusion cage of the invention;

FIG. 8 is a schematic diagram of a positional relationship between and structures of a first end face and a second end face of the intervertebral fusion cage of the invention; and

FIG. 9 is a top view of the structure of the intervertebral fusion cage of the invention.

### REFERENCE SIGNS

1 Intervertebral fusion cage
11 Filler hole
12 Side face
   121 First side face
   122 Second side face
   123 Third side face
   124 Fourth side face
13 Spacing
14 Pore area
   141 First pore area
   142 Second pore area
15 First end face
   151 First protrusion
16 Second end face
   161 Second protrusion
17 Instrument hole
   171 Instrument slot
18 Rounded corner
a Inclination angle
b Side face angle

### DETAILED DESCRIPTION

In order to make the object, technical solution and advantages of the invention clearer, the invention is further described in detail below with reference to the figures and in combination with the embodiments.

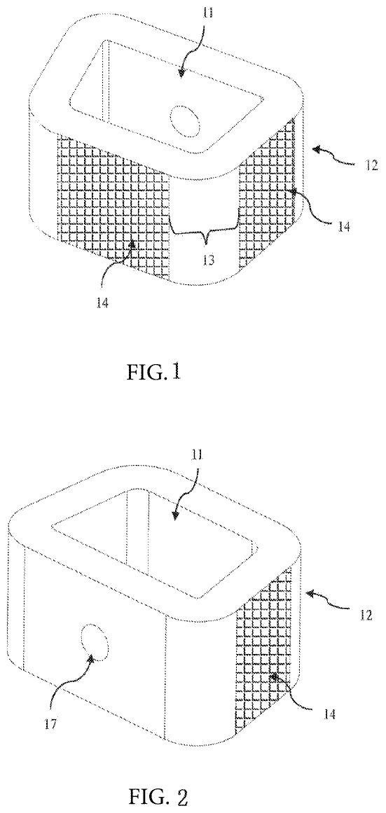

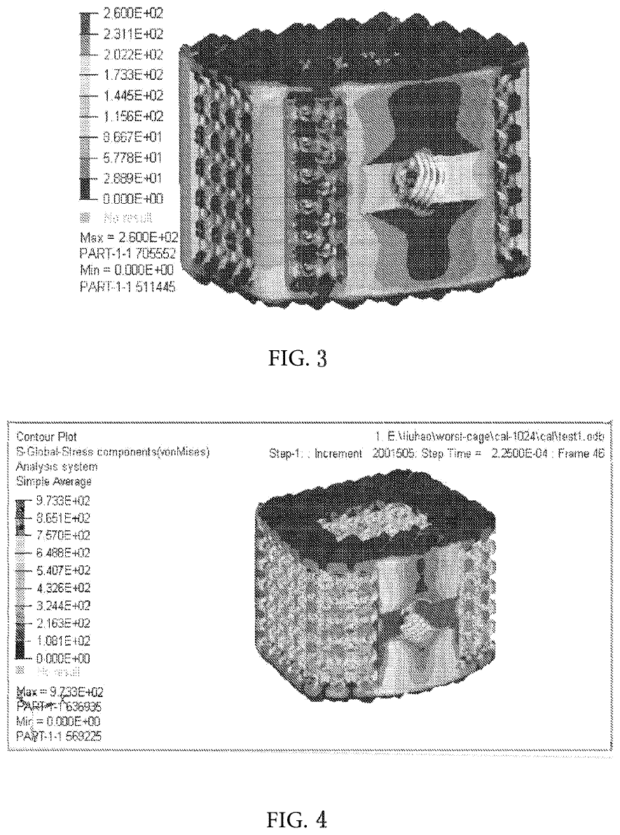

FIG. 1 is a schematic diagram of an overall structure of the intervertebral fusion cage of the invention in an embodiment, and FIG. 2 is a schematic diagram of an overall structure of the intervertebral fusion cage of the invention in another embodiment. FIG. 3 is a stress analysis diagram of an arrangement state 100 of pore areas of the intervertebral fusion cage, and FIG. 4 is a stress analysis diagram of an arrangement state 200 of pore areas of the intervertebral fusion cage. The arrangement state 100 shows a structure of a front end of the intervertebral fusion cage, in which the middle is a physical structure, pore structures are symmetrically distributed, and the left and right sides of the front end are provided with two supporting columns. The arrangement state 200 shows a structure of a front end of the intervertebral fusion cage, in which the middle is a physical structure, and pore structures are symmetrically distributed. As shown in FIG. 1 and FIG. 2 as well as FIG. 3 and FIG. 4, in an embodiment, the application provides an intervertebral fusion cage, and the intervertebral fusion cage 1 is provided with a filler hole 11, which runs through both ends of the intervertebral fusion cage 1; and

a plurality of pore areas 14 with spacings 13 are provided on a side face 12 of the intervertebral fusion cage 1.

In the embodiment, a specific structure of the intervertebral fusion cage 1 is provided. In the embodiment, a hexahedral structure is taken as an example of the intervertebral fusion cage 1, the both ends may be understood as two opposite faces in the hexahedral structure, and the filler hole 11, in which corresponding materials are filled to help an adherent growth of human tissues, runs through the both ends. In clinical operations, the both ends will support the vertebrae in the vertical direction, respectively, so the end faces of the both ends of the hexahedral structure cannot be provided with the pore areas 14, so as to prevent the end faces of the both ends from being fractured under the extrusion of the upper and lower vertebrae. There are a plurality of pore areas 14, which are arranged around the side face 12 of the intervertebral fusion cage 1, and there should be a spacing 13 between the adjacent pore areas 14 to ensure the strength of the side face 12 of the intervertebral fusion cage 1. It should be pointed out that the intervertebral fusion cage 1 being a hexahedral structure is an example in order to better illustrate the embodiment rather than a limitation of the embodiment.

The intervertebral fusion cage 1 is integrally formed, and may be made by 3D printing, and the material thereof is a titanium alloy. At present, polyether ether ketone (PEEK) is widely used in clinical practices. However, PEEK is not biologically active as the material of fusion cages, and cannot achieve a true fusion with upper and lower cartilage endplates, and most of surfaces are covered by fibrous tissues, which easily produces jiggles, and in turn affects a biomechanical stability between vertebral bodies, that is, which cannot ensure the stability of the overall structure. However, an intervertebral fusion cage made of a titanium alloy has a comparatively good biocompatibility and support strength.

In clinical practices, compression stiffness of a porous intervertebral fusion cage should be close to the stiffness of the human bones so as to reduce the stress shielding effect, and preferably, the stiffness is 30,000-100,000 N/mm.

A cuboid structure among the hexahedral structures may be also used in the intervertebral fusion cage 1, with the height range being preferably 6-10 mm, the length range being preferably 14-16 mm, and the width range being preferably 12-14 mm.

In FIG. 3 and FIG. 4, fixations are both made at bottom ends, and forces are both applied from the upper ends to simulate the real vertebral stress situation. Finally, FIG. 3 is the stress situation of the structure with the spacings 13 in the application, and in a case where all the other external conditions are consistent, the maximum stress value in FIG. 3 is 122 MPa, and the maximum stress value in FIG. 4 is 973 MPa. As may be seen, in the present application, thanks to the reasonable arrangement of the pore areas 14, the maximum stress borne by the intervertebral fusion cage 1 is greatly reduced.

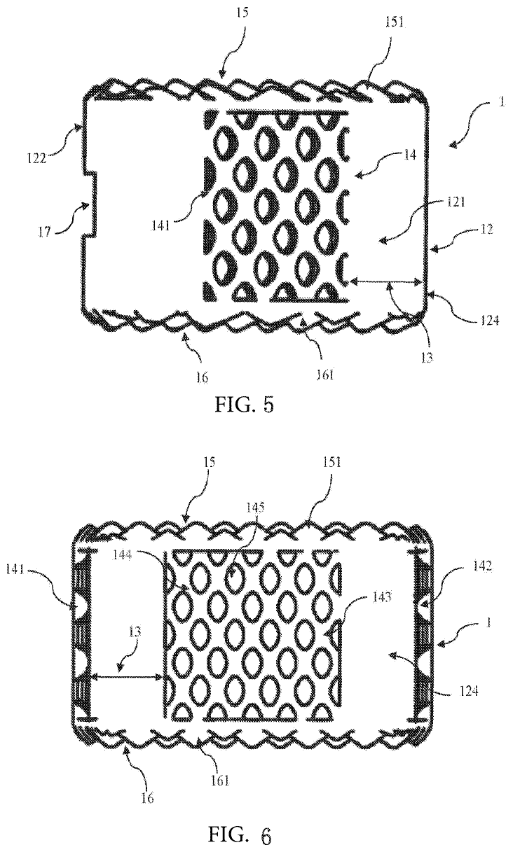

FIG. 5 is a schematic diagram of a side view of a structure of one side of the intervertebral fusion cage of the invention, and FIG. 6 is a schematic diagram of a front view of the structure of the intervertebral fusion cage of the invention. As shown in FIG. 5 and FIG. 6, in another embodiment, the both ends of the intervertebral fusion cage 1 are a first end face 15 and a second end face 16, respectively;

wherein at least one pore area 14 extends towards the both ends, and is connected to the first end face 15 and the second end face 16, respectively.

In the embodiment, a specific structure of the both ends is provided, and the arrangement relationship between the pore areas 14 and the both ends is further disclosed. The pore areas 14 are arranged along the circumference of the intervertebral fusion cage 1, and the spacings 13 are arranged in the surrounding direction of the plurality of pore areas 14 along the side face 12. It is disclosed in the embodiment the arrangement manner between the pore areas 14 and the both ends, that is, the first end face 15 and the second end face 16. Since the elastic modulus of the intervertebral fusion cage 1 mainly refers to one required by the first end face 15 and the second end face 16 for the reaction against the vertebrae when they are pressed against the intervertebral fusion cage 1 after being in contact with the vertebrae, respectively, so such arrangement aims to make the pore areas 14 provide the maximum elastic modulus adjustment amplitude between the first end face 15 and the second end face 16.

In an embodiment, the side face 12 is formed by successively connecting a first side face 121 and a second side face 122 as well as a third side face 123 and a fourth side face 124 and forming a closure;

the plurality of pore areas 14 include a first pore area 141 and a second pore area 142; and
the first pore area 141 and the second pore area 142 are provided on the first side face 121 and the third side face 123, respectively.

In the embodiment, an arrangement manner of the pore areas 14 on the intervertebral fusion cage 1 is specifically provided. In order to better explain the embodiment, the hexahedral structure is taken as an example, the side face 12 of the hexahedral structure includes the aforesaid four side faces, and it may be also understood that the first side face 121 is the left side face, the second side face 122 is the back face, the third side face 123 is the right side face, and the fourth side face 124 is the front face. In the embodiment, the left side face and the right side face are provided with a first pore area 141 and a second pore area 142, respectively, and the spacing 13 may be understood as an area between the first pore area 141 and the second pore area 142. In addition, the first pore area 141 and the second pore area 142 do not occupy the entire left and right side faces.

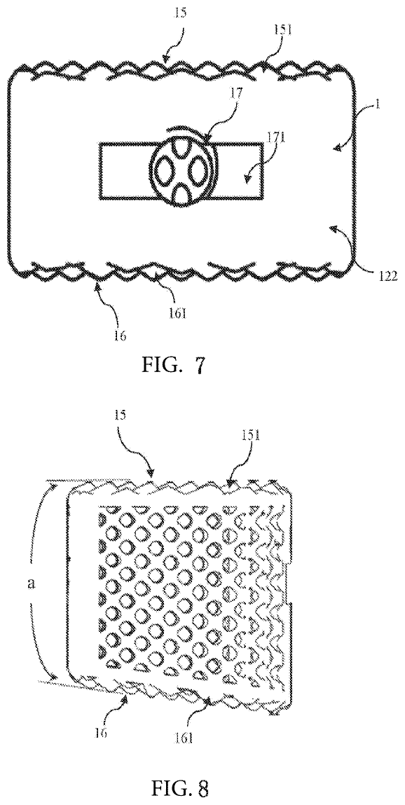

FIG. 7 is a schematic diagram of a rear view of a back face of the intervertebral fusion cage of the invention. As shown in FIG. 7, in an embodiment, the second side face 122 is provided with an instrument hole 17.

In the embodiment, a specific implementation mode of providing the second side face 122 with the instrument hole 17 is provided, an instrument slot 171 is further provided around the instrument hole 17, the instrument hole 17 and the instrument slot 171 are both provided for better docking with an external instrument, and the external instrument is used to place the intervertebral fusion cage 1 between the upper and lower vertebrae. The instrument hole 17 is provided on the side face 12 of the intervertebral fusion cage 1, and is specifically provided on the second side face 122, that is, on the back face, and the instrument hole 17 is provided towards the filler hole 11, so that the outside of the intervertebral fusion cage 1 communicates with the filler hole 11. In addition, most of the instrument holes 17 are threaded holes to be better connected to the external instruments. Although the threaded hole may be well connected to the external instrument, such structure will lead to stress concentration of the threaded hole, which is easily broken after being extruded by an external force. Especially after the pore areas 14 are arranged around the instrument hole 17, the pore areas 14 are often subjected to pressures from the upper and lower ends after being implanted in a human body, the pore areas 14 will be deformed to an extent, and such deformation will make the stress-concentrated threaded hole be fractured. In the embodiment, since there are very large distances between the left and right of the instrument hole 17 and the pore areas 14, and the instrument hole 17 is separately provided on the back face, so the pore areas 14 will not have an impact of stress concentration on the instrument hole 17, and when the pore areas 14 are subjected to an external force to be deformed, the impact on the instrument hole 17 will be also greatly reduced. In addition, the second side face 122 may be a protruding curved surface.

In an embodiment, the plurality of pore areas 14 further include a third pore area 143; and

the third pore area 143 is provided on the fourth side face 124.

In the embodiment, a specific implementation mode of providing the third pore area 143 on the fourth side face 124 is further provided, that is, the third pore area 143 is provided on the front face, the arrangement manner of the third pore area 143 is the similar to that of the first pore area 141 and the second pore area 142, and all of them may extend to the first end face 15 and the second end face 16. The implementation mode may further achieve an adjustment of the elastic modulus of the intervertebral fusion cage 1, without limitations to the achievement of the adjustment by means of the arrangement of the first pore area 141 and the second pore area 142.

In an embodiment, the first side face 121 and the second side face 122 as well as the third side face 123 and the fourth side face 124 transition by rounded corners 18.

In the embodiment, a specific implementation mode of the first side face 121 and the second side face 122 as well as the third side face 123 and the fourth side face 124 transitioning by the rounded corners 18 is provided. In the embodiment, the rounded corners 18 may be designed as the spacings 13, so that the pore areas 14 may extend longer along the circumference of the intervertebral fusion cage 1, and the spacings 13 may be further arranged, so that the structure of the intervertebral fusion cage 1 is more compact. In addition, the design of the rounded corners 18 also avoids the stress concentration. After all, the both sides of the rounded corner 18 will be connected to the pore areas 14, and if the pore areas 14 are deformed, the rounded corner 18 will be inevitably affected.

FIG. 8 is a schematic diagram of a positional relationship between and structures of a first end face and a second end face of the intervertebral fusion cage of the invention. As shown in FIG. 8, in an embodiment, there is an inclination angle “a” between the first end face 15 and the second end face 16.

In the embodiment, a specific implementation mode of there being the inclination angle “a” between the first end face 15 and the second end face 16 is provided, since the first end face 15 and the second end face 16 are attached to the upper and lower vertebrae, respectively, and all the vertebrae will have a physiological curvature after being arranged, when the intervertebral fusion cage 1 is placed between the upper and lower vertebrae, the inclination angle “a” between the first end face 15 and the second end face 16 may make the first end face 15 and the second end face 16 form a physiological curvature with the upper and lower vertebrae in terms of the structure. The inclination angle “a” preferably ranges from 0 to 7 degrees.

In an embodiment, a first protrusion 151 and a second protrusion 161 are provided on the first end face 15 and/or the second end face 16, respectively.

In the embodiment, it is provided that the first protrusion 151 and the second protrusion 161 are provided on the first end face 15 and/or the second end face 16, respectively. The first protrusion 151 and the second protrusion 161 are used for engagement with the vertebrae, which facilitates reduction of the movement of the intervertebral fusion cage 1 between the upper and lower vertebrae.

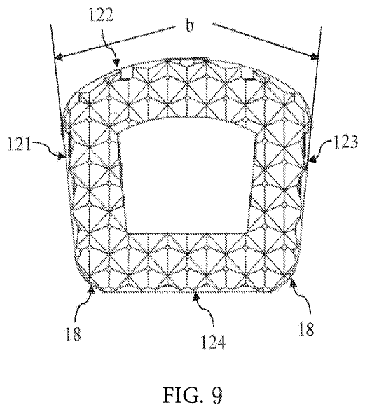

FIG. 9 is a top view of the structure of the intervertebral fusion cage of the invention. As shown in FIG. 9, in an embodiment, there is a side face angle “b” between the first side face 121 and the third side face 123.

In the embodiment, a specific implementation mode of there being the side face angle “b” between the first side face 121 and the third side face 123 is provided, so that the intervertebral fusion cage 1 is more in line with the shapes of the vertebrae.

In an embodiment, the pore area 14 is a crystal structure area with pores that is constructed by connecting rod supports.

In the embodiment, a specific structure of the pore area 14 is provided. The pore area 14 is a crystal structure area with pores 145 that is constructed by a plurality of connecting rods 144, and the rod diameter of the connecting rods 144 is preferably 100-800 μm. In the crystal structure area constructed by the both ends of the plurality of connecting rods 144 being connected to each other, the pores 145 between the connecting rods 144 after the connecting rods 144 construct the crystal structure area may be measured by an inscribed sphere, the diameter of the sphere is just the pore diameter, which is preferably 100-800 μm, and a ratio of a total space of the pores to a total space of the pore area 14 is called porosity, which is preferably 5%-90%.

The final elastic modulus of the pore area 14 is obtained after combining the rod diameter and the pore diameter as well as the porosity according to the expected elastic modulus.

It should be pointed out that the first pore area 141 and the second pore area 142 as well as the third pore area 143 may be understood as spatial structures constructed by the connecting rods 144 of different rod diameters being connected to each other, respectively so as to obtain pore areas with different elastic moduli. Similarly, pore areas with different elastic moduli may be also obtained by adjusting the pore diameter and the porosity.

The above embodiments are only preferred ones of the invention, and are not used to limit the invention. Any modifications, equivalent replacements, improvements, and so on made within the spirit and principle of the invention shall be included within the scope of protection of the invention.

1. An intervertebral fusion cage, characterized in that the intervertebral fusion cage (1) is provided with a filler hole (11), which runs through both ends of the intervertebral fusion cage (1); and
a plurality of pore areas (14) with spacings (13) are provided on a side face (12) of the intervertebral fusion cage (1).

2. The intervertebral fusion cage according to claim 1, characterized in that the both ends of the intervertebral fusion cage (1) are a first end face (15) and a second end face (16), respectively;
wherein at least one of the pore areas (14) extends towards the both ends, and is connected to the first end face (15) and the second end face (16), respectively.

3. The intervertebral fusion cage according to claim 1, characterized in that the side face (12) is formed by successively connecting a first side face (121) and a second side face (122) as well as a third side face (123) and a fourth side face (124) and forming a closure;
the plurality of pore areas (14) include a first pore area (141) and a second pore area (142); and
the first pore area (141) and the second pore area (142) are provided on the first side face (121) and the third side face (123), respectively.

4. The intervertebral fusion cage according to claim 3, characterized in that the second side face (122) is provided with an instrument hole (17).

5. The intervertebral fusion cage according to claim 4, characterized in that the plurality of pore areas (14) further include a third pore area (143); and
the third pore area (143) is provided on the fourth side face (124).

6. The intervertebral fusion cage according to claim 3, characterized in that the first side face (121) and the second side face (122) as well as the third side face (123) and the fourth side face (124) transition by rounded corners (18).

7. The intervertebral fusion cage according to claim 2, characterized in that there is an inclination angle (a) between the first end face (15) and the second end face (16).

8. The intervertebral fusion cage according to claim 2, wherein a first protrusion (151) and a second protrusion (161) are provided on the first end face (15) and/or the second end face (16), respectively.

9. The intervertebral fusion cage according to claim 3, characterized in that there is a side face angle (b) between the first side face (121) and the third side face (123).

10. The intervertebral fusion cage according to claim 1, characterized in that the pore area (14) is a crystal structure area with pores that is constructed by connecting rod supports.
